(19) United States
(12) Patent Application Publication
(10) Pub. No.: US 2009/0082766 A1
(43) Pub. Date: Mar. 26, 2009

(54) Tissue Sealer and End Effector Assembly and Method of Manufacturing Same

(75) Inventors: Jeffrey R. Unger, Longmont, CO (US); Robert M. Sharp, Boulder, CO (US); David W. Hixson, Longmont, CO (US); Chelsea Shields, Portland, OR (US); Darion Peterson, Boulder, CO (US); Jeremy James, Highlands Ranch, CO (US); David M. Garrison, Longmont, CO (US); Michael R. Warzecha, Longmont, CO (US); Edward M. Chojin, Boulder, CO (US); Duane E. Kerr, Berthoud, CO (US)

Correspondence Address: Tyco Healthcare Group LP, 60 MIDDLETOWN AVENUE, NORTH HAVEN, CT, 06473, US

(73) Assignee: TYCO Healthcare Group LP

(21) Appl. No.: 12/234,021

(22) Filed: Sep. 19, 2008

Related U.S. Application Data
(60) Provisional application No. 60/994,578, filed on Sep. 20, 2007.

(51) Int. Cl. A61B 18/14

(52) U.S. Cl. 606/51

(57) ABSTRACT

A bipolar forceps for sealing tissue includes at least one shaft having an end effector assembly disposed at a distal end thereof. The end effector assembly includes a pair of first and second opposing jaw members movable relative to one another from a first position wherein the jaw members are disposed in spaced relation relative to one another to a second position wherein the jaw members cooperate to grasp tissue therebetween. At least the first jaw member includes proximal and distal ends which define a cavity along a length thereof which houses an insulative member therein. The insulative member includes an electrically conductive sealing surface mounted thereto which resides in substantial opposition with a second electrically conductive sealing surface disposed on the second jaw member. One end of the first jaw member which defines the cavity extends a fixed distance toward the second jaw member to form a gap between electrically conductive surfaces when the jaw members are closed to grasp tissue.

### CROSS REFERENCE TO RELATED APPLICATION

This application claims the benefit of priority to U.S. Provisional Application Ser. No. 60/994,578 entitled “TISSUE SEALER AND END EFFECTOR ASSEMBLY AND METHOD OF MANUFACTURING SAME” filed Sep. 20, 2007 by Unger et al., the entire contents of which being incorporated by reference herein.

### BACKGROUND

The present disclosure relates to an electrosurgical instrument and method for performing electrosurgical procedures. More particularly, the present disclosure relates to an open or endoscopic bipolar electrosurgical forceps and method of manufacturing an end effector assembly having stop members associated with one or both of a pair of opposing jaw members. The stop members are designed to control the gap distance between opposing jaw members and enhance the manipulation and gripping of tissue during the sealing process.

### Technical Field

Forceps utilize mechanical action to constrict, grasp, dissect and/or clamp tissue. Electrosurgical forceps utilize both mechanical clamping action and electrical energy to effect hemostasis by heating the tissue and blood vessels. By controlling the intensity, frequency and duration of the electrosurgical energy applied through the jaw members to the tissue, the surgeon can coagulate, cauterize and/or seal tissue.

In order to effect a proper seal with larger vessels or thick tissue, two predominant mechanical parameters must be accurately controlled: the pressure applied to the tissue and the gap distance between the electrodes. As can be appreciated, both of these parameters are affected by the thickness of vessels or tissue. More particularly, accurate application of pressure is important for several reasons: to reduce the tissue impedance to a low enough value that allows enough electrosurgical energy through the tissue; to overcome the forces of expansion during tissue heating; and to contribute to the end tissue thickness, which is an indication of a good seal. It has been determined that fused tissue is optimum between about 0.001 inches to about 0.006 inches for small vessels and tissues and about 0.004 inches to about 0.010 inches for large, soft tissue structures. Below these ranges, the seal may shred or tear and above this range the tissue may not be properly or effectively sealed.

It is thought that the process of coagulating or cauterizing small vessels is fundamentally different than electrosurgical vessel or tissue sealing. “Vessel sealing” or “tissue sealing” is defined as the process of liquefying the collagen, elastin and ground substances in the tissue so that it reforms into a fused mass with significantly-reduced demarcation between the opposing tissue structures. In contrast, the term “cauterization” is defined as the use of heat to destroy tissue (also called “diathermy” or “electrodiathermy”) and the term “coagulation” is defined as a process of desiccating tissue wherein the tissue cells are ruptured and dried. Coagulation of small vessels is usually sufficient to permanently close them; however, larger vessels or tissue need to be “sealed” to assure permanent closure.

Numerous electrosurgical instruments have been proposed in the past for various open and endoscopic surgical procedures. However, most of these instruments cauterize or coagulate tissue and are normally not designed to provide uniformly reproducible pressure on the blood vessel or tissue which, if used for sealing purposes, would result in an ineffective or non-uniform seal. Other instruments generally rely on clamping pressure alone to procure proper sealing thickness and are often not designed to take into account gap tolerances and/or parallelism and flatness requirements, which are parameters that, if properly controlled, can assure a consistent and effective tissue seal.

Recently, instruments have been developed that utilize technology to form a vessel seal utilizing a unique combination of pressure, gap distance between opposing surfaces and electrical control to effectively seal tissue or vessels. Heretofore, a series of so-called stop members have been applied to the inner-facing, opposing tissue engaging surfaces to maintain a gap distance between opposing sealing surfaces of about 0.001 inches to about 0.010 inches. Typically, the stop members were sprayed atop the tissue engaging surfaces in various patterns by plasma deposition or other similar processes to assure proper parallelism when the jaw members were closed about tissue. In other instances, key-like gap plugs were employed to allow a user or manufacturer to selectively alter the size and shape of the stop members for a particular surgical purpose as described in U.S. Pat. No. 7,118,570. In yet other instances, a variable stop member is used that may be selectively adjusted to regulate the gap distance for particular tissue types and/or particular surgical purposes as described in U.S. patent application Ser. No. 10/846,262.

### SUMMARY

The present disclosure relates to a bipolar forceps for sealing which includes at least one shaft having an end effector assembly disposed at a distal end thereof. The end effector assembly has a pair of first and second opposing jaw members which are movable relative to one another from a first position wherein the jaw members are disposed in spaced relation relative to one another to a second position wherein the jaw members cooperate to grasp tissue therebetween. The first jaw member includes proximal and distal ends which define a cavity along a length thereof which houses an insulative member therein. The insulative member has an electrically conductive sealing surface mounted thereto that is positioned to reside in substantial opposition with a second electrically conductive sealing surface disposed on the second jaw member. At least one of the proximal and distal ends extends a fixed distance toward the second jaw member such that the end and the second jaw member form a gap between electrically conductive surfaces when the jaw members are closed to grasp tissue.

In one embodiment, the gap between electrically conductive surfaces is in the range of about 0.001 inches to about 0.010 inches. In another embodiment, the first electrically conductive sealing plate is connected to a first electrical potential from an electrosurgical energy source and the second electrically conductive sealing plate and both the first and second jaw members are connected to a second electrical potential from the electrosurgical energy source.

The present disclosure also relates to a method for manufacturing an end effector assembly for sealing tissue and includes the steps of: providing a pair of first and second jaw members each including an inwardly facing electrically conductive sealing surface; and coating the inwardly facing electrically conductive sealing surface of at least one of the jaw members with an insulative material having a thickness within the range of about 0.001 inches to about 0.010 inches. The electrically conductive sealing surface may include a knife channel defined therealong.

The method also includes the steps of: allowing the insulative material to cure onto the inwardly facing electrically conductive sealing surface; and trimming the insulative material from the inwardly facing electrically conductive sealing surface to form a series of stop members arranged thereacross. The pair of first and second jaw members is then assembled about a pivot such that the two inwardly facing electrically conductive sealing surfaces are substantially opposed to each other in pivotal relation relative to one another. The step of trimming may involve laser etching and the coating step may involve plasma deposition and/or pad printing.

The present disclosure also relates to a method for manufacturing an end effector assembly for sealing tissue and includes the initial step of providing a pair of first and second jaw members each having an outer insulative housing and an electrically conductive tissue sealing surface. The jaw members are moveable relative to one another from a first position wherein the jaw members are disposed in spaced relation relative to one another to a second position wherein the jaw members cooperate to grasp tissue therebetween. The method also includes the steps of disposing a series of insulative stop members atop the insulative housing of one (or both) jaw member and forming a corresponding series of apertures within the electrically conductive sealing plate of the jaw member in vertical registry with the stop members.

The method further includes the steps of: aligning the electrically conductive sealing plate of the jaw member atop the insulative housing such that each of the series of stop members are received through a respective aperture within the electrically conductive sealing plate; and securing the electrically conductive sealing plate of the jaw member atop the insulative housing of the jaw member such that the stop members project from the electrically conductive sealing plate a distance of about 0.001 inches to about 0.010 inches. The pair of jaw members is then assembled about a pivot such that the respective electrically conductive sealing surfaces are substantially opposed to each other in pivotal relation relative to one another.

The present disclosure also relates to a method for manufacturing an end effector assembly for sealing tissue and includes the steps of: providing a pair of first and second jaw members each having an electrically conductive tissue sealing surface and being moveable relative to one another from a first position wherein the jaw members are disposed in spaced relation relative to one another to a second position wherein the jaw members cooperate to grasp tissue therebetween. At least one of the electrically conductive tissue sealing surfaces of one of the jaw members includes a series of cavities defined therein. The method also includes the steps of: providing a substantially liquefied insulative material from a source; and dispersing an amount (e.g., a dollop) of the insulative material into at least one of the cavities to form a stop member which projects a distance of about 0.001 inches to about 0.010 inches from the electrically conductive tissue sealing surface.

The method further includes the steps of: allowing the insulative material to cure atop the electrically conductive sealing surface; and assembling the pair of first and second jaw members about a pivot such that the electrically conductive surfaces are substantially opposed to each other in pivotal relation relative to one another. In one particular embodiment, the series of cavities are generally key-shaped.

The present disclosure also relates to a method for manufacturing an end effector assembly for sealing tissue and includes the steps of: providing first and second electrically conductive sealing plates; encasing at least one of the sealing plates in a insulative material; applying a load to the sealing plates; melting the insulative material via a solvent or heat source; allowing a gap to form within the range of about 0.001 inches to about 0.010 inches between the sealing plates; and removing the heat source to allow the insulative material to cure.

The present disclosure also relates to a method for manufacturing an end effector assembly for sealing tissue and includes the steps of: providing first and second electrically conductive sealing plates; encasing at least one of the electrically conductive sealing plates in a substantially moldable insulative material; applying a load to the electrically conductive sealing plates; allowing the insulative material to deform to create a gap between the sealing plates between about 0.001 inches to about 0.010 inches; and allowing the insulative material to cure. The moldable insulative material may include a material that changes in density and/or volume upon application of heat, chemicals, energy or combinations thereof.

### BRIEF DESCRIPTION OF THE DRAWINGS

Various embodiments of the present disclosure are described herein with reference to the drawings wherein:

FIG. 1A is a right, perspective view of an endoscopic bipolar forceps according to the present disclosure having a housing, a shaft and a pair of jaw members affixed to a distal end thereof, the jaw members including an electrode assembly disposed therebetween;

FIG. 1B is a left, perspective view of an open bipolar forceps according to the present disclosure showing a pair of first and second shafts each having a jaw member affixed to a distal end thereof with an electrode assembly disposed therebetween;

FIG. 2 is a schematic, side view of a bipolar forceps according to an embodiment of the present disclosure having a recessed electrically conductive sealing surface that provides the requisite gap distance between sealing surfaces;

FIGS. 3A-3D are enlarged, top views showing one envisioned method of forming stop members on electrically conductive surfaces of a jaw member according to the present disclosure;

FIGS. 4A-4C are enlarged, perspective views showing another envisioned method of forming stop members on electrically conductive surfaces of a jaw member according to the present disclosure;

FIGS. 5A-5B is an enlarged, side view showing yet another envisioned method of forming stop members on electrically conductive surfaces of a jaw member according to the present disclosure; and

FIG. 6 is a flow diagram illustrating another method of manufacturing an end effector assembly according to the present disclosure;

### DETAILED DESCRIPTION

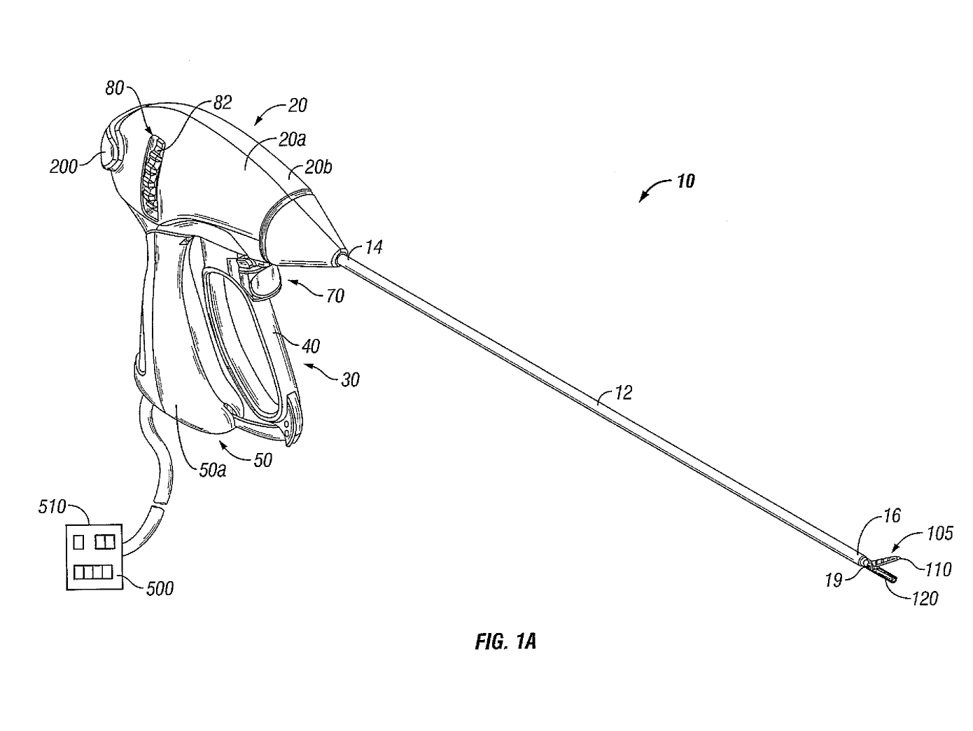

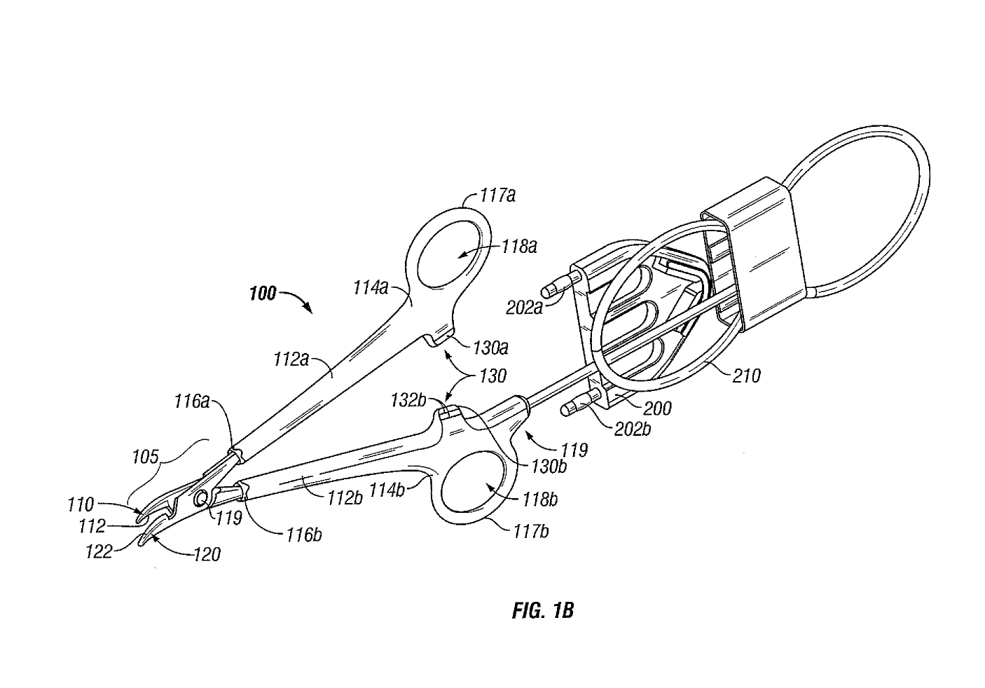

Referring now to FIGS. 1A and 1B, FIG. 1A depicts a bipolar forceps 10 for use in connection with endoscopic surgical procedures and FIG. 1B depicts an open forceps 100 contemplated for use in connection with traditional open surgical procedures. For the purposes herein, either an endoscopic instrument or an open instrument may be utilized with the end effector assembly described herein. Obviously, different electrical and mechanical connections and considerations apply to each particular type of instrument; however, the novel aspects with respect to the end effector assembly and its operating characteristics remain generally consistent with respect to both the open or endoscopic designs.

FIG. 1A shows a bipolar forceps 10 for use with various endoscopic surgical procedures and generally includes a housing 20, a handle assembly 30, a rotating assembly 80, a switch assembly 70 and an end effector assembly 105 having opposing jaw members 110 and 120 which mutually cooperate to grasp, seal and divide tubular vessels and vascular tissue. More particularly, forceps 10 includes a shaft 12 which has a distal end 16 dimensioned to mechanically engage the end effector assembly 105 and a proximal end 14 which mechanically engages the housing 20. The shaft 12 may include one or more known mechanically engaging components which are designed to securely receive and engage the end effector assembly 105 such that the jaw members 110 and 120 are pivotable relative to one another to engage and grasp tissue therebetween.

The proximal end 14 of shaft 12 mechanically engages the rotating assembly 80 (not shown) to facilitate rotation of the end effector assembly 105. In the drawings and in the descriptions which follow, the term “proximal”, as is traditional, will refer to the end of the forceps 10 which is closer to the user, while the term “distal” will refer to the end which is further from the user. Details relating to the mechanically cooperating components of the shaft 12 and the rotating assembly 80 are described in commonly-owned U.S. patent application Ser. No. 10/460,926 entitled “VESSEL SEALER AND DIVIDER FOR USE WITH SMALL TROCARS AND CANNULAS”.

Handle assembly 30 includes a fixed handle 50 and a movable handle 40. Fixed handle 50 is integrally associated with housing 20 and handle 40 is movable relative to fixed handle 50 to actuate the opposing jaw members 110 and 120 of the end effector assembly 105 as explained in more detail below. Movable handle 40 and switch assembly 70 are preferably of unitary construction and are operatively connected to the housing 20 and the fixed handle 50 during the assembly process. Housing 20 is preferably constructed from two components halves 20a and 20b which are assembled about the proximal end of shaft 12 during assembly. Switch assembly is configured to selectively provide electrical energy to the end effector assembly 105.

As mentioned above, end effector assembly 105 is attached to the distal end 16 of shaft 12 and includes the opposing jaw members 110 and 120. Movable handle 40 of handle assembly 30 imparts movement of the jaw members 110 and 120 from an open position wherein the jaw members 110 and 120 are disposed in spaced relation relative to one another, to a clamping or closed position wherein the jaw members 110 and 120 cooperate to grasp tissue therebetween.

Referring now to FIG. 1B, an open forceps 100 includes a pair of elongated shaft portions 112a and 112b each having a proximal end 114a and 114b, respectively, and a distal end 116a and 116b, respectively. The forceps 100 includes jaw members 120 and 110 which attach to distal ends 116a and 116b of shafts 112a and 112b, respectively. The jaw members 110 and 120 are connected about pivot pin 119 which allows the jaw members 110 and 120 to pivot relative to one another from the first to second positions for treating tissue. The end effector assembly 105 is connected to opposing jaw members 110 and 120 and may include electrical connections through or around the pivot pin 119. Examples of various electrical connections to the jaw members are shown in commonly-owned U.S. patent application Ser. Nos. 10/474,170, 10/116,824, 10/284,562 and 10/369,894, and U.S. Pat. Nos. 7,101,372, 7,083,618 and 7,101,371.

Each shaft 112a and 112b includes a handle 117a and 117b disposed at the proximal end 114a and 114b thereof which each define a finger hole 118a and 118b, respectively, therethrough for receiving a finger of the user. As can be appreciated, finger holes 118a and 118b facilitate movement of the shafts 112a and 112b relative to one another which, in turn, pivot the jaw members 110 and 120 from the open position wherein the jaw members 110 and 120 are disposed in spaced relation relative to one another to the clamping or closed position wherein the jaw members 110 and 120 cooperate to grasp tissue therebetween. A ratchet 130 is preferably included for selectively locking the jaw members 110 and 120 relative to one another at various positions during pivoting.

More particularly, the ratchet 130 includes a first mechanical interface 130a associated with shaft 112a and a second mating mechanical interface associated with shaft 112b. Each position associated with the cooperating ratchet interfaces 130a and 130b holds a specific, i.e., constant, strain energy in the shaft members 112a and 112b which, in turn, transmits a specific closing force to the jaw members 110 and 120. It is envisioned that the ratchet 130 may include graduations or other visual markings which enable the user to easily and quickly ascertain and control the amount of closure force desired between the jaw members 110 and 120.

As best seen in FIG. 1B, forceps 100 also includes an electrical interface or plug 200 which connects the forceps 100 to a source of electrosurgical energy, e.g., an electrosurgical generator (not shown). Plug 200 includes at least two prong members 202a and 202b which are dimensioned to mechanically and electrically connect the forceps 100 to the electrosurgical generator 500 (See FIG. 1A). An electrical cable 210 extends from the plug 200 and securely connects the cable 210 to the forceps 100. Cable 210 is internally divided within the shaft 112b to transmit electrosurgical energy through various electrical feed paths to the end effector assembly 105.

One of the shafts, e.g., 112b, includes a proximal shaft connector/flange 119 which is designed to connect the forceps 100 to the electrosurgical energy source 500. More particularly, flange 119 mechanically secures electrosurgical cable 210 to the forceps 100 such that the user may selectively apply electrosurgical energy as needed.

The jaw members 110 and 120 of both the endoscopic version of FIG. 1A and the open version of FIG. 1B are generally symmetrical and include similar component features which cooperate to permit facile rotation about pivot 19, 119 to effect the grasping and sealing of tissue. Each jaw member 110 and 120 includes an electrically conductive tissue contacting surface 112 and 122, respectively, which cooperate to engage tissue during sealing and cutting.

The various electrical connections of the end effector assembly 105 are preferably configured to provide electrical continuity to the electrically conductive tissue contacting surfaces 112 and 122 through the end effector assembly 105. For example, a series of cable leads may be configured to carry different electrical potentials to the conductive surfaces 112 and 122. Commonly owned U.S. patent applications Ser. Nos. 10/474,170, 10/116,824 and 10/284,562 all disclose various types of electrical connections which may be made to the conductive surfaces 112 and 122 through one or both of the shaft 112a and 112b. In addition, and with respect to the types of electrical connections that may be made to the jaw members 110 and 120 for endoscopic purposes, commonly-owned U.S. patent applications Ser. No. 10/369,894 and U.S. Pat. Nos. 7,101,372, 7,083,618 and 7,101,371 all disclose other types of electrical connections.

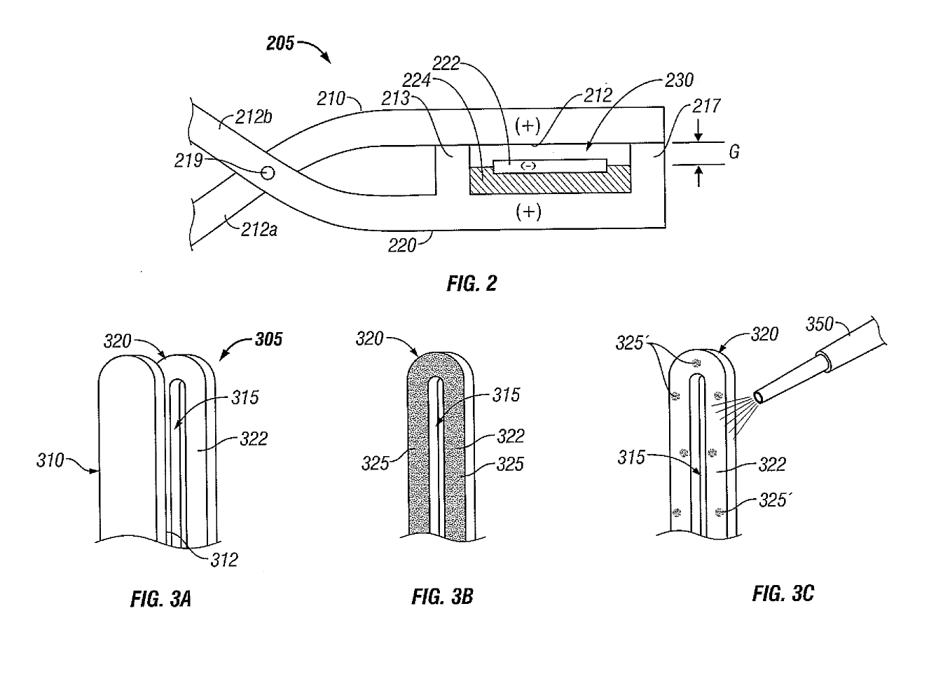

FIG. 2 shows one embodiment of an end effector assembly 205 for use with a bipolar forceps 10, 100 for sealing tissue that includes shafts 212a and 212b rotatable about a common pivot 219. The end effector assembly 205 has a pair of first and second opposing jaw members 210 and 220 that are selectively movable relative to one another from a first position wherein the jaw members 210, 220 are disposed in spaced relation relative to one another to a second position wherein the jaw members 210, 220 cooperate to grasp tissue therebetween. The first jaw member 220 includes a cavity or recess 230 defined therein that extends along a length thereof. The cavity 230 is dimensioned to house an insulative member 224 between respective proximal and distal ends 213 and 217. The insulative member 224 has an electrically conductive sealing surface 222 mounted thereto that is positioned to reside in substantial vertical opposition with a second electrically conductive sealing surface 212 disposed on the second jaw member 210.

Ends 213 and 217 of jaw member 220 extend a fixed distance toward the second jaw member 210 such that the ends 213 and 217 and the second jaw member 210 form a gap “G” between electrically conductive surfaces 212 and 222 when the jaw members 210 and 220 are closed to grasp tissue. As mentioned above, two mechanical factors play an important role in determining the resulting thickness of the sealed tissue and effectiveness of a tissue seal, e.g., the pressure applied between opposing jaw members 210 and 220 and the gap distance “G” between the opposing tissue contacting surfaces 212 and 222 during the sealing process. With particular respect to vessels and small tissue bundles, a gap distance “G” during sealing within the range of about 0.001 inches to about 0.010 inches is particularly suitable for effectively sealing tissue. Other gap ranges may be preferable with other tissue types such as bowel or large vascular structures. A working pressure within the range of about 3 kg/cm² to about 16 kg/cm² between sealing surfaces 212 and 222 has been shown to be effective for sealing various tissue types.

Electrically conductive sealing surface 222 is coupled to a first electrical potential from an electrosurgical energy source, e.g., generator 500 (see FIG. 1A), and sealing plate 212 and jaw members 220 are coupled a second electrical potential from the electrosurgical energy source. In use, tissue is initially grasped between jaw members 210 and 220 and positioned within cavity 230. The shaft members 212a and 212b are pivoted to close the jaw members 210 and 220 about the tissue under a pressure within the above working range. As mentioned above, ends 213 and 217 are dimensioned to maintain a gap distance “G” between the sealing surfaces 212 and 222 such that upon activation, electrosurgical energy travels between the different electrical potentials to form an effective tissue seal between sealing surfaces 212 and 222. Jaw member 220 may be configured such that only one end, e.g., proximal end 213, is dimensioned to maintain the requisite gap distance between sealing surfaces 212 and 222.

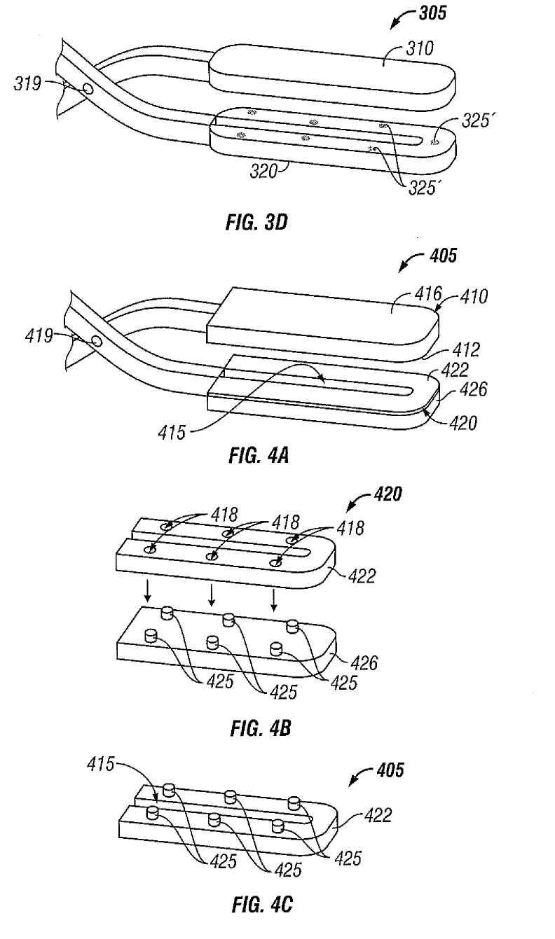

FIGS. 3A-3D show one method for manufacturing an end effector assembly 305 for sealing tissue according to the present disclosure and includes the initial step of providing a pair of jaw members 310 and 320 each including an inwardly facing electrically conductive sealing surface 312 and 322. The method also includes the steps of: coating the inwardly facing electrically conductive sealing surface 322 of at least one of the jaw members, e.g., jaw member 320, with an insulative material or substrate 325 having a thickness within the range of about 0.001 inches to about 0.010 inches; and allowing the insulative material to cure onto the inwardly facing electrically conductive sealing surface 322. Once cured, the method includes the step of trimming the insulative material 325 from the inwardly facing electrically conductive sealing surface 322 to form a series of stop members 325′ arranged thereacross. A laser 350 (or other suitable etching or removal tool) may be utilized to etch or form the stop members 325′. The pair of first and second jaw members 310 and 320 are then assembled about a pivot 319 such that the two inwardly facing electrically conductive sealing surfaces 312 and 322 are substantially opposed to each other in pivotal relation relative to one another.

In one embodiment, the step of trimming may involve laser etching and the coating step may involve plasma deposition and/or pad printing. One or both of the electrically conductive sealing surfaces 312 and 322 may include a knife channel defined therealong for reciprocating a knife (not shown) therein for cutting tissue.

FIGS. 4A-4C show yet another method for manufacturing an end effector assembly 405 for sealing tissue according to the present disclosure and includes the initial step of providing a pair of first and second jaw members 410 and 420 each having an outer insulative housing 416 and 426 and an electrically conductive tissue sealing plate 412 and 422, respectively. The jaw members 410 and 420 are moveable relative to one another about a pivot 419 from a first position wherein the jaw members 410 and 420 are disposed in spaced relation relative to one another to a second position wherein the jaw members 410 and 420 cooperate to grasp tissue therebetween. The method also includes the steps of disposing a series of insulative stop members 425 atop an insulative substrate of at least one of the jaw members, e.g., jaw member 420, and forming a corresponding series of apertures 418 within the electrically conductive sealing plate 422 of the jaw member 420 in vertical registry with the stop members 425.

The method further includes the steps of: aligning the electrically conductive sealing plate 422 of the jaw member 420 atop the insulative substrate 426 such that each of the series of stop members 425 is received through a respective aperture 418 within the electrically conductive sealing plate 422; and securing the electrically conductive sealing plate 422 atop the insulative substrate 426 such that the stop members 425 project from the electrically conductive sealing plate 422 a distance within the range of about 0.001 inches to about 0.010 inches. The pair of jaw members 410 and 420 is then assembled about pivot 419 such that the respective electrically conductive surfaces 412 and 422 are substantially opposed to each other in pivotal relation relative to one another.

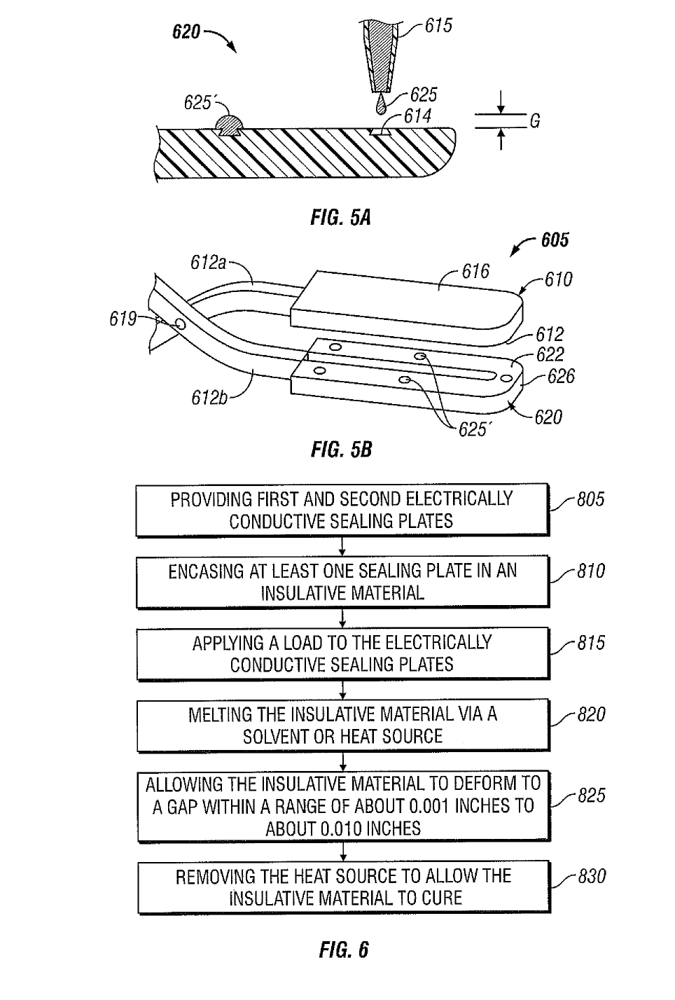

FIGS. 6A and 5B show yet another method for manufacturing an end effector assembly 605 for sealing tissue according to the present disclosure and includes the initial step of providing a pair of first and second jaw members 610 and 620 each having an electrically conductive tissue sealing surface 612 and 622, respectively. The jaw members 610 and 620 are moveable relative to one another from a first position wherein the jaw members 610 and 620 are disposed in spaced relation relative to one another to a second position wherein the jaw members 610 and 620 cooperate to grasp tissue therebetween. At least one of the electrically conductive tissue sealing surfaces, e.g., surface 622, includes a series of cavities 614 defined therein. The method also includes the steps: of providing a substantially liquefied insulative material 625 from a source of liquefied insulative material 615; and dispersing an amount (e.g., a dollop) of the insulative material 625 into at least one of the cavities 614 of to form a stop member 625′ that projects a distance of about 0.001 inches to about 0.010 inches from the electrically conductive tissue sealing surface 622.

The method further includes the steps of: allowing the stop member 625′ to cure atop the electrically conductive sealing surface 622 and assembling the pair of first and second jaw members about a pivot 619 such that the electrically conductive surfaces 612 and 622 are substantially opposed to each other in pivotal relation relative to one another In one particular embodiment, the series of cavities 614 are generally key-shaped. Other suitable geometric shapes are also envisioned that will provide secure engagement of the stop member 625′ atop the sealing surface 622 once cured, e.g., polygonal, t-shaped, I-beam, etc.

FIG. 6 illustrates another method for manufacturing an end effector assembly for sealing tissue (not shown) and includes the initial step 805 of providing first and second electrically conductive sealing plates. Step 810 includes encasing at least one sealing plate in an insulative material. Step 815 includes applying a load to the electrically conductive sealing plates and step 820 includes melting the insulative material via a solvent or heat source. Step 825 includes allowing the insulative material to deform to a gap within a range of about 0.001 inches to about 0.010 inches between sealing plates. Step 830 includes removing the heat source to allow the insulative material to cure. One or both jaw members may be manufactured in this fashion and then assembled to create an end effector assembly for use with sealing tissue.

Another method according to the present disclosure relates to a method for manufacturing an end effector assembly for sealing tissue and includes the steps of: providing first and second electrically conductive sealing plates; encasing at least one of the electrically conductive sealing plates in a substantially moldable insulative material; applying a load to the electrically conductive sealing plates; allowing the insulative material to deform to create a gap between the sealing plates between about 0.001 inches to about 0.010 inches; and allowing the insulative material to cure. The moldable insulative material may include a material that changes in density and/or volume upon application of heat, chemicals, energy or combinations thereof.

From the foregoing and with reference to the various figure drawings, those skilled in the art will appreciate that certain modifications can also be made to the present disclosure without departing from the scope of the same. For example, forceps 10, 100 or any of the aforedescribed end effector assemblies 105, 305, 405, 505 or 605 may be designed such that the assembly is fully or partially disposable depending upon a particular purpose or to achieve a particular result. More particularly, end effector assembly 105 may be selectively and releasably engageable with the distal end 16 of the shaft 12 and/or the proximal end 14 of the shaft 12 may be selectively and releasably engageable with the housing 20 and handle assembly 30. In either of these two instances, the forceps 10 would be considered “partialy disposable” or “reposable”, i.e., a new or different end effector assembly 105 (or end effector assembly 105 and shaft 12) selectively replaces the old end effector assembly 105 as needed.

An insulator (not shown) may also be included to limit and/or reduce many of the known undesirable effects related to tissue sealing, e.g., flashover, thermal spread and stray current dissipation. At least one of the electrically conductive surfaces, e.g., 322, of one of the jaw members, e.g., 320, includes a longitudinally-oriented channel 315 defined therein (See FIG. 3A) that extends from the proximal end of the electrically conductive seating surface 322 to the distal end. The channel 315 facilitates longitudinal reciprocation of a knife (not shown) along a preferred cutting plane to effectively and accurately separate the tissue along a formed tissue seal.

By controlling the intensity, frequency and duration of the electrosurgical energy applied to the tissue, the user can selectively seal tissue. The generator 500 may include a controller 510 (See FIG. 1A) that operatively couples to one or more sensors (not shown) that determine or measure tissue thickness, tissue moisture, tissue type, tissue impedance, etc. and automatically signal the controller 510 to adjust the electrosurgical energy prior to or during the sealing process to optimize the tissue seal.

The stop member(s) may be dimensioned in any suitable geometric configuration and may be disposed on or adjacent to one or both of the electrically conductive tissue sealing surfaces or operatively associated with one or both jaw members.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A bipolar forceps for sealing tissue, comprising:
at least one shaft having an end effector assembly disposed at a distal end thereof, the end effector assembly including a pair of first and second opposing jaw members movable relative to one another from a first position wherein the jaw members are disposed in spaced relation relative to one another to a second position wherein the jaw members cooperate to grasp tissue therebetween; and
at least the first jaw member including proximal and distal ends that define a cavity along a length of the first jaw member;
an insulative member disposed within the cavity, the insulative member including an electrically conductive sealing surface mounted thereto disposed in substantial opposition to a second electrically conductive sealing surface disposed on the second jaw member; and
wherein at least the proximal end of the first jaw member extends a fixed distance toward the second jaw member such that the proximal end and the second jaw member form a gap between electrically conductive surfaces when the jaw members are in the second position.

2. A bipolar forceps according to claim 1 wherein both the proximal end of the first jaw member and the distal end of the first jaw member extend toward the second jaw member to form the gap between electrically conductive surfaces when the jaw members are in the second position.

3. A bipolar forceps according to claim 1 wherein the gap between electrically conductive surfaces is in the range of about 0.001 inches to about 0.010 inches.

4. A bipolar forceps according to claim 1 wherein the first electrically conductive sealing plate is connected to a first electrical potential from an electrosurgical energy source and the second electrically conductive sealing plate and both the first and second jaw members are connected to a second electrical potential from the electrosurgical energy source.
